SEQUENCE LISTING (4 sequences)

SEQ ID NO: 1 - protein, 6 aa, synthetic construct
HHHHHH

SEQ ID NO: 2 - protein, 10 aa, synthetic construct
EQKLISEEDL

SEQ ID NO: 3 - protein, 11 aa, synthetic construct
DSLEFIASKLA

SEQ ID NO: 4 - protein, 16 aa, synthetic construct
GSGSHHWGSTHHHHHH

(19) United States
(12) Patent Application Publication
(10) Pub. No.: US 2024/0384221 A1
(43) Pub. Date: Nov. 21, 2024

(54) Compositions and Methods For Semi-Automated Protein Production

(71) Applicant: University of Washington, Seattle, WA (US)

(72) Inventors: David Baker, Seattle, WA (US); Lukas F. Milles, Seattle, WA (US); Basile I.M. Wicky, Seattle, WA (US)

(21) Appl. No.: 18/658,657

(22) Filed: May 8, 2024

Related U.S. Application Data
(60) Provisional application No. 63/464,881, filed on May 8, 2023.

(51) Int. Cl. C12M 1/36; B01D 15/34; C07K 1/16; C12N 15/70; C12P 21/00; G01N 35/00; G01N 35/10

(52) U.S. Cl. CPC C12M 41/48; B01D 15/34; C07K 1/16; C12N 15/70; C12P 21/00; G01N 35/0099; G01N 35/10

(57) ABSTRACT

Provided are systems, modules, compositions, and methods comprising integrated and modular workflows effective for high-throughput production and characterization of target polypeptides cost-effective protein production on the milligram scale, enabling characterization of hundreds of designs per day in a standardized format.

### CROSS-REFERENCE TO RELATED APPLICATIONS

This application claims the benefit of U.S. Provisional Application Ser. No. 63/464,881, filed May 8, 2023, which is incorporated by reference herein in its entirety.

### REFERENCE TO AN ELECTRONIC SEQUENCE LISTING

This application contains a Sequence Listing that has been submitted electronically and is hereby incorporated by reference in its entirety. The Sequence Listing was created on Aug. 6, 2024, is named “24-0595-US_SequenceListing_ST26.xml”, and is 4,592 bytes in size.

### FIELD

The disclosure relates to the high throughput design, production, and characterization of polypeptides. More particularly, the disclosure provides integrated, semi-automated high-throughput systems, compositions, modules, and methods for designing, generating, purifying, and characterizing targeted polypeptides in a rapid, modular, scalable, and cost-effective production stream that allows for standardized preparation and characterization of hundreds of polypeptide targets per day.

### BACKGROUND

Recombinant protein expression and purification followed by biochemical characterization are cornerstones of protein engineering research, including de novo synthesis and target validation screening. Recent advances in de novo protein design have vastly outpaced standard protein biochemistry protocols, making experimental validation a significant bottleneck in such methods. Typical workflows are resource intensive (time, cost, and labor), often requiring multiple weeks for testing a few dozen designs. Moreover, existing workflows typically lack protocol standardization and consistent data collection, thus precluding large-scale and systematic investigations of the parameter space influencing design success. Pooled assays can test large numbers of designs in parallel (e.g., thousands or even millions) and provide a compelling alternative to individual design testing when large amounts of data are required for a project. Nevertheless, pooled assays are usually highly specialized towards measuring only one property (e.g. binding or enzymatic activity), require significant upfront protocol development, and rarely provide information on the manufacturability of individual sequences (expression, solubility, dispersity, etc.). Accordingly, the field would benefit significantly from a simple assay-agnostic workflow for the production and initial characterization of hundreds of proteins per day in a standardized, cost-effective manner.

This disclosure provides integrated, semi-automated high-throughput systems, compositions, modules, and methods that significantly accelerate target protein design, production, validation, and characterization.

### SUMMARY OF THE DISCLOSURE

In an aspect, the disclosure relates to a semi-automated protein production platform comprising a polyclonal expression system that comprises a plurality of cloning products, wherein each of the plurality of the cloning products comprises a gene encoding a protein target and a background suppression vector, wherein the plurality of cloning products are adapted for transformation to generate recombinant cells, and wherein the generated recombinant cells are capable of direct expression of the protein targets.

In some embodiments, the semi-automated protein production platform further comprises a laboratory robot and/or a liquid handling device. In yet further embodiments, the laboratory robot or liquid handling device comprises an OT-2 robot, an autopipettor, a robotic pipettor, or an acoustic liquid handler.

In some embodiments, the semi-automated protein production platform further comprises at least one chromatography column selected from an affinity column, an ion exchange column, a hydrophobic interaction column, a size exclusion column, or any combination of two of more thereof.

In some of the above embodiments, the semi-automated protein production platform further comprises a mass spectrometer.

In another aspect, the disclosure relates to a semi-automated protein production method comprising, in a continuous workflow sequence: identifying at least one target protein sequence to be produced by the method; preparing at least one cloning reaction wherein the at least one cloning reaction comprises constructing a target gene sequence from one or more DNA sequences that encodes the at least one target protein sequence, in combination with a vector that comprises background suppression, to generate at least one cloning reaction product; transforming cells with the at least one cloning reaction product from the at least one cloning reaction to generate a recombinant cell; and inducing the recombinant cell to express the target protein.

In some embodiments, the method further comprises purifying the target protein from the recombinant cell. In embodiments, purifying the target protein comprises at least one of an affinity column, an ion exchange column, a hydrophobic interaction column, a size exclusion column, or any combination of two of more thereof.

In some embodiments, the method further comprises determining the molecular weight of the target protein. In embodiments, determining the molecular weight of the target protein comprises at least one of size exclusion chromatography, mass spectrometry, or gel electrophoresis.

In some of the above embodiments, the method can further comprise determining the concentration of the purified target protein. In yet further embodiments, the method can further comprise dispensing an amount of the purified target protein into a vessel, wherein the amount of purified protein that is dispensed is in a volume that provides for a normalized concentration.

In an aspect, the disclosure relates to a non-transitory computer-readable medium having computer-executable instructions stored thereon that, if executed by one or more processors of a computing device, cause the computing device to perform operations comprising:
controlling a laboratory robot to apply a respective sample from each of a plurality of wells of one or more multi-well sample plates to an input port of a device or instrument, wherein each target protein of a set of target proteins corresponds to one or more respective samples within at least one of the plurality of wells, and wherein each well of the plurality of wells is associated with at least one expected measurable characteristic or value associated with the respective target protein;
operating the device or instrument to determine a characteristic or value associated with the protein for the at least one of the plurality of wells;
comparing each of the measured characteristic or value to the corresponding expected measurable characteristic or value to determine a subset of the plurality of wells that contain successfully produced target protein; and
operating the laboratory robot to transfer, from the subset of the plurality of wells, respective amounts of protein into respective wells of a target multi-well sample plate such that each protein of a target set of proteins is transferred into at least one respective well of the target sample plate.

In embodiments of this aspect, the operating of the device or instrument comprises a size exclusion chromatography instrument to determine respective measured protein molecular weights for each of the plurality of wells. In yet further embodiments, the size exclusion chromatography instrument is operated such that samples from more than one of the plurality of wells are resident within the size exclusion chromatography instrument at a time. In some other embodiments, operating the size exclusion chromatography instrument further comprises determining respective measured protein yields and/or concentrations for each of the plurality of wells, and wherein operating the laboratory robot further comprises transferring from the one or more wells respective amounts of protein into a particular well of the target multi-well sample plate such that an amount and/or a concentration of protein in the particular well corresponds to a target concentration.

In some further embodiments, the operating of the size exclusion chromatography instrument determines respective measured protein yields for each of the plurality of wells comprises, for each of the plurality of wells, based on determining an integrated absorbance at 280 nanometers.

In some embodiments of the above aspects, the operating of the device or instrument comprises a mass spectrometer to determine respective measured protein molecular weights for each of the plurality of wells.

In another aspect, the disclosure relates to a non-transitory computer-readable medium having computer-executable instructions stored thereon that, if executed by one or more processors of a computing device, cause the computing device to perform operations comprising:
operating a laboratory robot and/or liquid handling device to prepare one or more cloning reactions by dispensing a respective sample from each of a plurality of wells to form one or more reaction volumes, wherein each of the one or more reaction volumes corresponds to at least one target protein sequence, and wherein the samples forming the reaction volume are dispensed from (i) at least one of the plurality of wells comprising one or more DNA sequences that encodes the at least one target protein sequence, and (ii) at least one of the plurality of wells comprising a background suppression vector, to generate at least one cloning reaction product in each of the one or more reaction volumes;
operating a laboratory robot and/or liquid handling device to transfer a volume of competent cells to each of the one or more reaction volumes comprising the cloning reaction product to form a transformation mixture;
maintaining the transformation mixture under conditions that allow for transformation of the competent cells to generate recombinant cells comprising the cloning reaction product; and
maintaining the recombinant cells under conditions that allow for the recombinant cell to express the target protein.

In some embodiments of this aspect, the operations are effective to maximize the yield of the one or more cloning reactions for a correct target plasmid and/or to reduce background plasmids comprising a failed cloning reaction. In some further embodiments, the operations effective to maximize the yield of a cloning reaction one or more cloning reactions comprise selecting (i) a background suppression vector comprising a ccdb lethal gene and/or (ii) the one or more DNA sequences comprise overhangs designed for simultaneous and directional assembly of the DNA sequences into a single DNA sequence using a Type IIS restriction enzyme and T4 DNA ligase, and optionally verifying products of the cloning reactions by at least one of DNA sequencing or mass spectrometry.

Other aspects and embodiments falling within the scope and spirit of the disclosure will be apparent to those of skill in the art in light of the following description and illustrative examples.

### BRIEF DESCRIPTION OF DRAWINGS

FIG. 1 depicts the general workflow in accordance with the various aspects and embodiments of the disclosure. Starting from protein sequence identification/selection (100) the workflow proceeds in an orchestrated and direct manner starting with the design and synthesis of DNA sequences for gene construction (110). Once the linear DNA fragments needed for gene construction are obtained (120), the method proceeds to gene synthesis (130) and plasmid assembly, with direct transformation of cells (140) for the subsequent expression of the target proteins (150). Once sufficient protein is generated, the target proteins are isolated and purified from the cells (160), and are pooled at determined concentrations for further characterization (170). As described herein, the assembly of linear DNAs to construct the genes and transformed cells through protein expression constitute the polyclonal methodology that provides substantial advantages over current high throughput de novo protein synthesis methods. Notably, this high throughput workflow can take as little as about 42-45 hours from gene synthesis to purified target proteins.

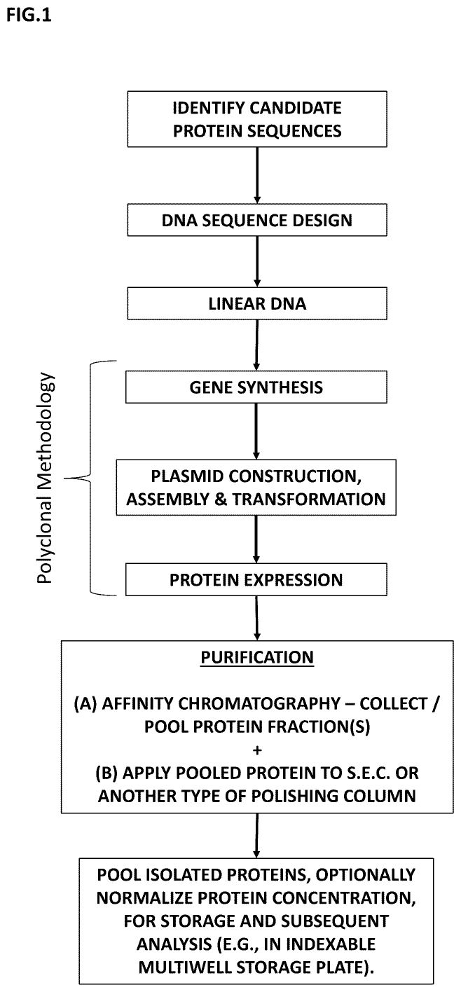

FIG. 2A-2C depicts (A) Representative result of a SAPP run of 96 samples, generated in accordance with the embodiments of the disclosure, that showing chromatograms of all size exclusion chromatography (SEC) traces, ordered by hierarchical clustering. (B) Depicts yields and aggregation state overview for the 96 proteins purified in accordance with the embodiments of the disclosure. (C) Demonstrates that the high throughput workflows in accordance with the embodiments of the disclosure are reproducible, depicting parity plots of biological replicates of the same 96 protein designs showing analyzed total yield, and position of main elution peak.

FIG. 3A-3D depicts (A) Overview of resulting data for another SAPP run of 96 protein samples generated in accordance with the embodiments of the disclosure, showing SEC runs on the left as an overlay and also offset by index on the right. (B) Corresponding summary for the 96 samples from (A) showing a gradient for yield from 4 mL expression cultures of every sample plotted as retention volume against expected molecular weight, with calibration of the SEC column (Cytiva S75 5/150) shown in gray and open circles. (C) Technical replicates for the dataset shown in (A) and (B) conducted by two different experimenters on different days. Parity plots for each replicate showing key metrics demonstrate the reliability of the high throughput methods in total protein yield from 4 mL culture, SEC main peak elution volume, SEC main peak Absorbance at 280 nm, normalized peak retention of main elution peak (all gradient coded by total protein yield). (D) Clustered SEC chromatography results for preparations of 576 different protein samples, collected by a single experimenter over 6 days.

FIG. 4 depicts an illustrative system in accordance with some of the aspects and embodiments of the disclosure.

FIG. 5 depicts an illustrative flowchart of a set of operations that can be performed by a system in accordance with some aspects and embodiments of the disclosure.

### DETAILED DESCRIPTION

The disclosure generally provides systems and methods that includes a modular workflow for medium to large scale protein production (“Semi-Automated Protein Production” or “SAPP”), that combines throughput-designed molecular biology methods, open-source robotics, and an automated analysis pipeline to purify and characterize hundreds of proteins designs per day that only requires an individual to spend a few active hours of total time at the bench. Once the underlying DNA sequences are obtained, complete end-to-end execution of the production is completed within 48 h, enabling rapid iteration cycles. Typical protein yields are sufficient for downstream analysis and assays (e.g. next generation sequencing, mass spectrometry, binding assays, enzymatic activity assays, cell assays, electron microscopy, etc.). Advantageously, the data generated by the workflow is standardized and consolidated into an open format that enables quantitative analyses between designs and their experimental properties.

In broad overview, the workflow starts from a set of candidate amino acid sequences and, preferably, creates DNA sequences from automated reverse-translation that maximize synthesizability and codon adaptation, and can add adapters for downstream cloning into a suite of compatible plasmids for multiplexed cloning (a polyclonal methodology). The same design can be subcloned into multiple vectors depending on the desired application(s) (e.g. His-tag, Strep-tag, Avi-tag, NanoBiT, Halo-tag, Maltose Binding Protein, Fluorescent Proteins, (see, e.g., Table 1 for a list 50 illustrative vectors that have been deposited at Addgene)). Large constructs (1.5-3 kb) are automatically split into two compatible fragments for one-pot assembly. The DNA constructs can be prepared as linear gene fragments and/or ordered from commercial manufacturers, and cloned into the desired receiving vector(s) using Golden Gate Assembly (GGA), followed by direct transformation into an E. coli expression strain. Target vectors are designed with background suppression which is leveraged to rapidly identify and determine whether the gene is inserted successfully during GGA, and avoid the drawbacks associated with the state of the art techniques (e.g., validation by colony picking and sequencing). As demonstrated herein, the process from linear DNA to inoculated cultures can take as little as two hours for a total of 192 reactions.

While polyclonality arising from synthesis errors could be expected to present a risk with regard to the applicability of the method, surprisingly, sequencing analysis of 1973 GGA reactions showed a clonal purity over 98.6%. Mass spectrometry analysis of 863 purified proteins also revealed the expected mass for 91% of products, with no or undetectable levels of contamination for all expression products, including mostly insoluble or aggregated protein. These results demonstrate that the polyclonal methodology can save time and resources by circumventing the standard approach of picking clones and sequencing in cases where trading a few dropouts for greater throughput is advantageous.

Thus, in an aspect the disclosure provides for a rapid, cost-effective semi-automated protein production system that screen target protein designs on a medium throughput scale (e.g., 96-192 proteins per run), that leverages a polyclonal methodology using parallelized GGA cloning of linear DNA fragments that are directly transformed into recombinant cells for expression, purification, and characterization. As noted above, the polyclonal methodology increases the efficiency, time, and resources required to take target protein designs from concept to purified protein. The systems and methods described herein also provide an extended library of premade GG target vectors designed with compatible GG overhangs, so that the same linear fragment can be cloned flexibly into vectors with different tags or fusions generally available in protein expression technology (e.g., HIS-tags, AviTag for SPR, to GFP and force spectroscopy handle fusions). The polyclonal step and transformation is followed directly by small scale expression in 96 well plates (4 mL, autoinduction media) and purification (e.g., chromatographic methods as described herein), and characterization. The automated methods described herein can also surprisingly increase the speed of size exclusion chromatography experiments, wherein a separation can be achieved in less than 5 minutes per sample (96 in 7.5 hours, on Cytiva Superdex 5-150 columns) while retaining resolution to separate oligomeric species, and providing sufficient yields for downstream biophysical analyses—all within five to seven days of DNA design, and with three or fewer hours of bench time required of a researcher. The automated SAPP method described herein can synergistically increase experimental throughput by creating efficiencies at each processing step by avoiding the labor intensive and error-prone steps typically performed by researchers. The procedure can be executed end-to-end from a single notebook using custom code to interface with the different sections of the protocol, such as automatic generation of scripts for programming robotic steps and data analysis. All data generated during the process is contained in a single dataframe to make runs comparable, and build up databases of protein sequences and associated biochemical labels.

### Definitions

All headings are for the convenience of the reader and should not be used to limit the meaning of the text that follows the heading, unless so specified.

As used herein and unless otherwise indicated, the terms “a” and “an” are taken to mean “one”, “at least one” or “one or more” (i.e., can indicate a plurality). As such, and unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

Unless defined otherwise, all technical and scientific terms used herein have the same meaning as commonly understood by one of ordinary skill in the art to which the disclosure belongs. A number of terms and abbreviations appear throughout the disclosure and, unless otherwise indicated, should be understood to have the definitions that follow.

A “nucleic acid” or “polynucleotide” sequence are used herein to refer to deoxyribonucleotides or ribonucleotides and polymers thereof in either single-or double-stranded form. The terms encompass nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The terms “amino acid sequence,” “polypeptide,” “protein,” and “peptide” as used herein all refer to a sequence of amino acid residues linked by peptide bonds. The amino acid sequence can be of any length of greater than two amino acids. In some embodiments proteins can be de novo designed sequences. Polypeptides can include modified forms of the sequence, such as naturally occurring or synthetically generated post-translational modifications, or modifications to the chemical structure of one or more amino acid residues. Non-limiting examples of modified forms include glycosylated sequences, phosphorylated sequences, myristoylated sequences, palmitoylated sequences, ribosylated sequences, acetylated sequences, and the like. Modifications can also include intra-or inter-molecular crosslinking or covalent attachments to moieties such as lipids, flavin, biotin, polyethylene glycol or derivatives thereof, and the like. In addition, modifications may also include protein cyclization, branching of the amino acid chain, and cross-linking of the protein. Further, amino acids other than the naturally-encoded twenty amino acids may also be included in a polypeptide.

One or three letter codes for amino acids are used herein. For example, alanine is A or Ala, arginine is R or Arg, asparagine is N or Asn, aspartic acid is D or Asp, asparagine or aspartic acid is B, cysteine is C or Cys, glutamic acid is E or Glu, glutamine is Q or Gln, glutamine or glutamic acid is Z, glycine is G or Gly, histidine is H or His, isoleucine is I or Ile, leucine is L or Leu, lysine is K or Lys, methionine is M or Met, phenylalanine is F or Phe, proline is P or Pro, serine is S or Ser, threonine is T or Thr, tryptophan is W or Trp, tyrosine is Y or Tyr, valine is V or Val.

The protein sequences can be isolated and/or purified, both of which refer to a protein that is substantially separated from other cellular components (i.e., host cell proteins (HCP), DNA, RNA, lipids, membranes, cell debris) of the organism in which the sequence is produced (e.g., 30%, 40%, 50%, 60%, 70%, 80%, 90%, 95%, 96%, 97%, 98%, 99%, 100% free of contaminants).

A “recombinant” nucleic acid or amino acid sequence is a nucleic acid or protein/polypeptide produced by recombinant DNA technology. The terms “recombinant,” “heterologous,” and “exogenous,” can be used interchangeably herein and, when referring to polynucleotides, mean a polynucleotide (e.g., a DNA or RNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, the terms when referring to polypeptides, means a polypeptide or amino acid sequence that originates from a source foreign to the particular host cell or, if from the same source, is modified from its original form. As such, recombinant DNA molecules can be expressed in a host cell to produce a recombinant polypeptide.

The terms “transformed,” “transgenic,” and “recombinant,” when used with reference to cells typically refer to an isolated cell or a cell in culture, such as a microbial (e.g. bacterial or yeast) cell, into which a coding sequence or gene has been introduced or a heterologous amino acid sequence is expressed. Typically the gene is incorporated into the cell on an extrachromosomal molecule (e.g., plasmid, expression vector).

The terms “plasmid,” “vector,” and “cassette” (e.g., transformation cassette, expression vector, or expression cassette) generally refer to an extra-chromosomal element that comprises nucleic acid sequences (e.g., genes, promoters, regulatory elements (inducers, repressors, etc.) and the like) which are not part of the central metabolism of the cell, and can be circular, double-stranded DNA molecules. Such elements may be autonomously replicating sequences, genome integrating sequences, phage or nucleotide sequences (linear or circular) of a single-or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Merely for purposes of clarity, a “transformation cassette” refers to a vector containing a foreign gene and having elements in addition to the foreign gene that facilitate transformation of a particular host cell. An “expression cassette” or “expression vector” refers to a vector containing a foreign gene and having elements in addition to the foreign gene that allow for expression and/or enhanced expression of that gene in a foreign host. Thus, the terms can refer to a nucleic acid molecule capable of transporting another nucleic acid molecule to which it has been linked. A non-limiting example of an expression vector includes a gene encoding a target protein with a promoter that is functional in bacteria, where the promoter and gene are oriented such that the promoter drives expression of the target protein in the bacterial cell.

A “linker” refers to a short amino acid sequence that separates multiple domains of a polypeptide. In some embodiments, the linker prohibits energetically or structurally unfavorable interactions between the discrete domains.

A recombinant gene can be “codon optimized” when its nucleotide sequence is modified to accommodate codon bias of the host organism, typically to improve gene expression and increase translational efficiency of the gene.

As used herein a “coding sequence” generally refers to a DNA sequence that encodes for a specific amino acid sequence.

A “regulatory sequence” is generally used to refer to a polynucleotide sequence located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

A “promoter” refers to a DNA sequence capable of controlling the expression of a coding sequence or functional RNA. Commonly, a coding sequence is oriented or located 3′ to a promoter sequence. Promoters may be derived in their entirety from a native gene, or be composed of different elements derived from different natural promoters, or comprise synthetic DNA segments. Different promoters may direct the expression of a gene in different cell types, or at different stages of development or cell growth/cycle, or in response to different environmental conditions. Promoters that cause a gene to be expressed in most cell types at most times are commonly referred to as “constitutive promoters.”

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” or “over-expression” generally refers to the transcription and stable accumulation of sense (mRNA) or antisense RNA derived from a nucleic acid sequence that is generated in recombinant cells.

“Transformation” is used according to its ordinary and customary meaning as understood by a person of ordinary skill in the art, and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “recombinant” or “transformed” organisms.

In one aspect, the disclosure relates to a semi-automated protein production system comprising one or more modules, methods, compositions and systems as described herein.

In another aspect, the disclosure relates to a semi-automated protein production device comprising one or more modules, methods, compositions and systems as described herein.

In a further aspect, the disclosure relates to a semi-automated protein production method comprising one or more steps, modules, compositions and systems as described herein.

In another aspect, the disclosure relates to a semi-automated protein production composition comprising one or more modules, methods, compositions and systems as described herein.

In another aspect, the disclosure relates to a non-transitory computer-readable medium having computer-executable instructions stored thereon that, if executed by one or more processors of a computing device, cause the computing device to perform one or more steps for semi-automating protein production as described herein.

In yet further aspects, the disclosure provides a semi-automated protein production platform comprising a polyclonal expression system that comprises a plurality of cloning products, wherein each of the plurality of the cloning products comprises a gene encoding a protein target and a background suppression vector, wherein the plurality of cloning products are adapted for transformation to generate recombinant cells, and wherein the generated recombinant cells are capable of direct expression of the protein targets.

In some further aspects, the disclosure provides a method for semi-automated protein production comprising, in a continuous workflow sequence, preparing a plurality of cloning reactions wherein each of the plurality of cloning reactions comprises preparing a target gene sequence in combination with a vector that comprises background suppression; transforming cells with products from the plurality of cloning reactions to generate a plurality of recombinant cells; and inducing the plurality of recombinant cells to express the target protein.

In particular embodiments of the above aspects, the polyclonal expression systems and methods in accordance with the disclosure improve on state of the art methods and production platforms at least by increasing the efficiencies associated with the time, fidelity, and resources of typical methodologies. For example, the modules and methods described herein all for target protein production without the typical time-intensive cloning strategies that are representative of the state of the art, such as performing DNA sequencing on cloning products in order to confirm accuracy of the sequence. The polyclonal techniques and compositions described herein provide surprising advantages in that they can reduce time, cost, and inefficiencies associated with the typical de novo protein design sequence validation. As discussed above, sequencing analysis of nearly two thousand cloning reactions performed in accordance with the disclosure demonstrated a clonal purity over 98.6%. These results demonstrate that the polyclonal methodology can save time and resources by circumventing the standard approach of picking clones and sequencing in cases where trading only a few dropouts for greater throughput is advantageous. The polyclonal techniques and compositions in accordance with the disclosure have been demonstrated to have low rates of both synthesis errors and failed cloning reactions, and are particularly efficient in light of the total number of cloning reactions that the methods can generate.

In embodiments, the polyclonal method comprises a plurality of cloning reactions to generate cloning products (i.e., plurality of cloned genes in plasmids), wherein each cloning reaction comprises preparing a target gene sequence in combination with a background suppression vector, transforming the cloning products (i.e., plasmids) from the cloning reactions to generate recombinant cells and inducing protein expression without confirming accuracy of the target sequence by, e.g., DNA sequencing. In a majority of the polyclonal cloning reactions, the method provides for cloning products that include the correct construct (i.e., target protein sequence) as the dominant component of the expression culture following its direct transformation. In various further embodiments, the construct and protein product can be verified by any number of methods effective in determining protein sequence and/or molecular weight such as, for example, gel electrophoresis, size exclusion chromatography, Multi-Angle Light Scattering, Mass Photometry, analysis of protease fragmentation reactions, mass spectrometry, native mass spectrometry, immunological techniques (e.g., specific binding proteins, antibodies, ELISA, western blot, etc.), Nanopore protein sequencing.

In some embodiments the background suppression vector allows for methods and compositions that provide for one or more selection marker. As used herein, “selection marker” has broad meaning and can include any type of marker that is useful in identifying the presence or absence of the marker. Some non-limiting examples of a selection marker include a lethal gene (e.g., toxins), a resistance and/or selection drug (e.g., antibiotic resistance, colormetric, etc.), an essential gene (e.g., a compensatory essential gene or transcription factor in a knockout or defective cell type or cell line), and others generally known in the art.

In yet further embodiments, the background suppression vector comprises a lethal gene, such as the non-limiting examples of the ccdb gene or colicin genes or lysis genes. As used herein, a “lethal gene” can be any gene that provides for background suppression of a failed cloning reaction. For example, in some embodiments of the disclosure transformants comprising sequences or plasmids that lack the cloned gene sequence will carry an undisrupted and expressed lethal gene that kills the undesired transformed cells. In some particular embodiments, a background suppression vector or can be compatible with specific restriction enzymes and/or incorporate specific DNA overhang sequences that provide for convenient and high fidelity cloning reactions. In some further embodiments, background suppression vector can be an entry vector and can be compatible with the Bsal or any other TypeIIs restriction enzyme such as BsmBI, SapI, BbsI, PaqCI and/or incorporate AGGA/TTCC overhangs or other overhangs if required. In yet further embodiments, the entry vectors comprise a pBR322 origin of replication (medium-copy). Some non-limiting examples of entry vectors are provided in Table 1, some of which have been submitted to and are available at the Addgene nonprofit plasmid on-line repository at addgene.org.

TABLE 1
Illustrative entry vectors.

| | | | Affinity | | |
|---|---|---|---|---|---|
| Name | Resist | Prom. | tag | N-term fusion | C-term fusion |
| LM565 | Kan | T7 | His | | |
| LM567 | Kan | T7 | His | | His-tag |
| LM627 | Kan | T7 | His | | (SNAC-tag)-(His-tag) |
| LM668 | Kan | T7 | His | GLP ybbr-tag)- | GS linker-Fg gamma pept. |
| | | | | (His-tag) | |
| LM670 | Kan | T7 | His | | His-tag |
| LM671 | Kan | T7 | His | sfGFP | His-tag |
| LM673 | Kan | T7 | | sfGFP-(SNAC-tag) | |
| LM1369 | Kan | T7 | | | |
| LM1371 | Kan | T7 | His | His-tag | |
| LM1423 | Kan | T7 | | | |
| LM1425 | Kan | T7 | His | | (Avi-tag)-(His-tag) |
| LM1426 | Kan | T7 | His | | (His-tag)-SpyTag003 |
| LM1487 | Kan | T7 | His | (His-tag)-mScarlet-I | |
| LM1488 | Kan | T7 | His | (His-tag)-TagGFP2 | |
| LM1489 | Kan | T7 | His | (His-tag)-MTagBFP2 | |
| LM1490 | Kan | T7 | | mScarlet-I | |
| LM1491 | Kan | T7 | | TagGFP2 | |
| LM1493 | Kan | T7 | | mNeonGreen | |
| LM1494 | Kan | T7 | His | | |
| LM1496 | Kan | T7 | | TagCFP | |
| LM1498 | Kan | T7 | | sfmTurquoise2 | |
| LM1499 | Kan | T7 | | mKelly2 | |
| LM1701 | Kan | T7 | | | |
| BW1001 | Kan | T7 | His | | |
| BW1002 | Kan | T7 | His | | |
| BW1003 | Kan | T7 | His | | |
| BW1004 | Kan | T7 | His | (His-tag)-smBiT | |
| BW1005 | | T7 | His | (His-tag)-GB1-smBiT | |
| BW1006 | | T7 | His | (His-tag)-IgBiT | |
| BW1007 | | T7 | His | | His-tag |
| BW1008 | Amp | T7 | His | | SNAC-(His-tag) |
| BW1009 | | T7max | His | | SNAC-(His-tag) |
| BW1010 | Amp | T7max | His | | SNAC-(His-tag) |
| BW1011 | | T7 | His | His-tag | CfaN |
| BW1012 | | T7 | His | GB1-CafC | His-tag |
| BW1013 | | T7 | His | | CafN-(His-tag) |
| BW1014 | | T7 | His | (His-tag)-GB1-CfaC | |
| BW1015 | | T7 | His/2 | | CafN |
| BW1016 | | T7 | His/2 | GB1-CfaC | |
| BW1017 | | T7max | His | | His-tag |
| BW1018 | | T7max | His | His-tag | |
| BW1019 | | T7max | His | (His-tag)-GB1-smBiT | |
| BW1020 | | T7max | His | (His-tag)-IgBiT | |
| BW1021 | | T7max | His | | |
| BW1022 | Amp | T7 | His | | His-tag |
| BW1023 | Amp | T7 | His | | |
| BW1024 | Amp | T7 | His | His-tag | |
| BW1025 | Amp | T7 | His | (His-tag)-GB1-smBiT | |
| BW1026 | Amp | T7 | His | (His-tag)-IgBiT | |
| BW1027 | Amp | T7max | His | | His-tag |
| BW1028 | Amp | T7max | His | | |
| BW1029 | Amp | T7max | His | His-tag | |
| BW1030 | Amp | T7max | His | (His-tag)-GB1-smBiT | |
| BW1031 | Amp | T7max | His | (His-tag)-IgBiT | |
| DT0001 | Amp | T7 | Strep II | | Strep-II-tag |
| KT0001 | Amp | T7 | His | GB1-smBiT | IgBiT |
| KT0002 | Amp | T7 | His | IgBiT | SmBiT-GB1-(His-tag) |
| AL0001 | Amp | T7 | | | |
| PL1337 | Amp | T7 | His | GB1 | His-tag |
| WY0001 | Amp | T7 | His | (Myc-tag)-(His-tag) | |

POI is protein of interest; (His-tag) is a hexa-histidine (HHHHHH, SEQ ID NO: 1) tag for purification; (SNAC-tag) is a sequence-specific nickel-assisted cleavage tag; (Myc-tag) is the sequence (EQKLISEEDL, SEQ ID NO: 2) from human c-Myc protein; (ybbr-tag) is sequence (DSLEFIASKLA, SEQ ID NO: 3) from the ybbR ORF gene of Bacillus subtilis; sfGFP is superfolder green fluorescent protein variant; IgBiT is the large bit of the NanoLuc splitluciferase; smBiT is the small bit of the NanoLuc splitluciferase; GB1 is Protein G from Streptococci, used as a solubility and/or mass tag; CafC is C terminal region of split intein Cfa; CafN is N terminal region of split intein Cfa; mKelly2 is a fluorescent protein; sfmTurquoise2 a fluorescent protein; TagCFP is a fluorescent protein; mNeonGreen is a fluorescent protein; mScarlet-I is a fluorescent protein; MTagBFP2 is a fluorescent protein; MTagGFP2 is a fluorescent protein; and SpyTag is a peptide tag that can form a covalent bond with its partner spycatcher.

### DNA, Plasmids, and Recombinant Cells

In some embodiments, the semi-automated protein production systems, modules, methods, and compositions comprise DNA sequences and/or methods of designing and/or synthesizing DNA sequences that encode a target protein sequence. The DNA sequences can be determined by any method known in the art. In some preferred embodiments, the DNA sequences are converted from the target protein sequence using an automated executable script, rather than manual conversion from codon usage tables. In embodiments, a script is capable of any one or more of generating sequences that (i) are codon-optimized for expression in multiple organisms (E. Coli, human, yeast, etc.), (ii) prevent alternative start sites in the genes, and (iii) are optimized for the synthesis of the DNA sequence. In some preferred embodiments any necessary or desired DNA overhangs for cloning reactions are included in the DNA sequence prior to synthesis either on a DNA synthesizer or as an output file for a third party DNA manufacturer/supplier. In some embodiments, the script can automatically execute a number of quality checks (e.g., no duplicate sequences, sequence length for multi-fragment assembly, synthesizability of sequences using manufacturer API).

Nucleic acid (i.e., DNA) sequences in accordance with the disclosure are synthesized and cloned using techniques known in the art. Gene expression can be controlled by inducible or constitutive promoter systems using the appropriate plasmids and/or expression vectors. Cells are transformed using standard transformation methods and compositions but which are modified for use in high throughput format to generate recombinant cells.

The DNA sequences can be used in, or used to generate, modified recombinant cells, which produce target protein sequences. In some embodiments, the nucleic acid sequence (i.e., DNA) encoding a gene or a complementary nucleic acid sequence to such a coding sequence can be codon optimized for production in a selected microorganism. A number of factors can be used in determining a codon-optimized sequence and can include, for example, (1) selecting a codon for each amino acid residue in the recombinant polypeptide based on the usage frequency of each codon in the heterologous host cell (e.g., E. coli, S. cerevisiae, etc.) genome; (2) removing sequences that provide for restriction sites for enzymes to prevent DNA cleavage; (3) modifying long repeats (e.g., consecutive sequences of 5 or more nucleotide) to prevent low-complexity regions; (4) adding a ribosome binding site to the N-terminus; (5) adding a stop codon; (6) changing nucleotides that encode amino acids susceptible to undesirable post-translational modifications (e.g., changing codons for a surface exposed LYS to an ARG codon to avoid ubiquitination); (7) removing or replacing a localization signal sequence.

In various embodiments, the DNA sequences can further comprise additional sequence encoding amino acids that are not part of the sequence of the target proteins. In some these embodiments, the additional sequences encode additional amino acids present when the nucleic acid is translated, encoding, for example, an additional protein domain, for example to generate a tagged or a fusion protein. Other examples of additional sequences can include flexible linker sequences, localization sequences (e.g., directing the protein to a specific subcellular compartment or membrane), affinity tags (e.g., 6xhis tag), cleaving sequences (e.g., SNAC, protease recognition sequences, self-cleaving sequences, etc.), affibody tags, localization scaffolds, vacuolar localization tags, co-folding domains, and/or secretion signals, or combinations thereof.

In some embodiments, the semi-automated protein production systems, modules, methods, and compositions comprise plasmids and/or methods of designing and/or synthesizing plasmids for expressing a gene encoding a target protein sequence. In embodiments, the plasmids can comprise nucleotide sequences that are not translated. Non-limiting examples include promoters, terminators, barcodes, Kozak sequences, targeting sequences, and enhancer elements. In some embodiments the plasmids comprise a promoter that is functional in a particular host cell (e.g., yeast, fungi, and/or bacteria). In embodiments, expression of a gene encoding a target protein is controlled by the promoter operably linked to the gene sequence. For a gene to be expressed, a promoter must be present within 1,000 nucleotides upstream of the gene. A gene is generally cloned under the control of a desired promoter. The promoter is placed upstream of the gene in the genome or on an episomal plasmid. The promoter regulates the amount of protein expressed in the cell and the timing of expression, or expression in response to external factors such as carbon source.

Any promoter can be utilized to drive the expression of the target proteins described herein. Various promoters for various organisms (e.g., bacteria and yeast) are known and readily available.

As discussed above, various embodiments of the disclosure provide the DNA sequence as an expression cassette, e.g., a plasmid. Any plasmid/expression cassette capable of expressing the protein in a host cell can be utilized. Additional regulatory elements can also be present in the expression cassette, including restriction enzyme cleavage sites, antibiotic resistance genes, integration sites, auxotrophic selection markers, origins of replication, and degrons. In embodiments, the plasmid comprises one or more of the exemplary plasmids described in Table 1.

In some embodiments, expression cassette can be present in a vector that either integrates into chromosomal DNA or remains episomal in the recombinant host cell. A variety of such vectors are well-known in the art, including non-limiting examples of a yeast vectors such as a yeast episomal plasmid (YEp) that contains the pBluescript II SK (+) phagemid backbone, an auxotrophic selectable marker, yeast and bacterial origins of replication and multiple cloning sites enabling gene cloning under a suitable promoter. Other non-limiting vectors include pRS series plasmids.

As described above, some embodiments in accordance with the disclosure provide plasmids for expressing proteins that are constructed from synthetic DNA according to a method wherein linear DNA fragments encoding target protein design sequences, and including overhangs that are suitable for a restriction digest (e.g., BsaI) are cloned into custom target vectors (e.g., Golden Gate Assembly). In embodiments entry vectors can be created to support a variety of N-and C-terminal tags. In some embodiments, a standard expression vector used for screening designs can include C-terminally HIS-tagged constructs: MSG-design-GSGSHHWGSTHHHHHH (SEQ ID NO: 4, entry vector LM627), where the underlined sequence is the SNAC-tag used for cleaving the HIS-tag, and with a TRP residue to ensure proteins have measurable absorbance at 280 nm.

### Protein Expression

In accordance with the aspects and embodiments disclosed herein, the systems and methods provide for mid-to high throughput expression of proteins. In some embodiments, the methods and devices can use minimized sample volumes, such as such as microvolumes or nanovolumes. Accordingly, aspects of the disclosure relate to methods, systems, and devices for assembly/cloning of gene sequences encoding target protein sequences and protein expression in small volumes and/or droplets on separate and addressable features of a support (reaction wells, support surface(s), and the like). In some embodiments, predefined reaction microvolumes on the nanoliter and microliter scales may be used. However, smaller or larger volumes may be used. One of skill will appreciate that the minimized sample volume can increase the number of samples that can be processed in an efficient and parallel manner. For example, the expression reaction can take place on a solid surface or support, such as a microwell plate or array. In some embodiments, the expression reactions can be performed on the same support as used for the gene/plasmid assembly. Yet in other embodiments, the transcription and expression reactions are performed in separate reaction vessels (e.g., on separate supports).

In embodiments protein expression is performed with recombinant/transformed cells that are grown in expression or induction media (as generally known in the art), either from freshly transformed cells, or freezer stocks. In some embodiments, cultures for protein expression can seeded from a growth plate (e.g., LB plate) or directly from the recovered transformants in induction media, and the cultures are maintained under conditions that are effective for protein production as are known in the art (e.g., under appropriate aeration, temperature, time, supplements/feeds, etc.).

### Protein Purification

Once expressed, the proteins produced in accordance with the systems and methods of the disclosure can be purified according to standard procedures known in the art, (e.g., precipitation, ion-exchange chromatography, affinity chromatography, hydrophobic interaction chromatography, size exclusion chromatography, electrophoresis, and the like (see, generally, Scopes, R., Protein Purification, Springer-Verlag, N.Y. (1982)). Once purified to the degree desired, the proteins can be used in and/or further characterized by any assays or methods that are known in the art. In some embodiments, proteins can be purified by affinity tag-based chromatographic methods. In yet further embodiments, proteins can be purified by a combination of chromatographic methods (e.g., HIS tag-immobilized metal affinity chromatography (IMAC) and S75 or S200 size exclusion chromatography). In some embodiments, the amounts of purified target protein are sufficient for routine protein characterization and/or functional assays such as, for example, solubility/thermal stability, mass spectroscopy, force spectroscopy and kinetic binding assays such as Fluorescence Polarization, Biolayer Interferometry, or Surface Plasmon Resonance, structural analyses such as negative-stain electron microscopy and cryo electron microscopy.

In yet some further embodiments, the size exclusion chromatography (SEC) data can processed through an interactive Jupyter notebook, which consolidates all recorded data and calculates the expected properties of expressed proteins, such as molecular weights. From SEC trace peaks at defined retention volumes the oligomeric state of the proteins can be determined. The SEC trace peaks also can be used to determine protein yield and/or concentration by integrating the absorbance at 280 nm. In some further embodiments, analysis software compares the measured molecular weights (from SEC, assigned by running known calibration standards, Cytiva LMW and HMW calibration kits) with the expected values and selects appropriate fractions for pooling. As detailed below, executable instructions can control a device, such as an OT-2 robot, to combine selected column fractions into a single 96 or 384 well plate and, optionally, can normalize concentrations using the integrated A280 absorbance data from the SEC traces. In such embodiments, the disclosure can provide a series of samples in a multiwall plate that contain all target protein designs at known concentrations and with all associated data. All information and experimental data on the target protein designs that are obtained or tested is consolidated in a single dataframe that can be used for downstream analysis.

### Recombinant host cells

In an aspect the disclosure provides recombinant cells that comprise the polynucleotides, plasmids, and/or proteins described herein. In embodiments, the host cells can comprise any type of cell that is adaptable to genetic manipulation and/or expression of foreign genes and proteins. In embodiments, host cells may be any species of bacteria, including but not limited to Escherichia, Corynebacterium, Caulobacter, Pseudomonas, Streptomyces, Bacillus, or Lactobacillus. In some embodiments, the host cell is a yeast cell such as, for example, Saccharomyces, Candida, Pichia, Schizosaccharomyces, Scheffersomyces, Blakeslea, Rhodotorula, or Yarrowia.

These cells can achieve gene expression controlled by inducible promoter systems, natural or induced mutagenesis, recombination, and/or shuffling of genes, pathways, and whole cells performed sequentially or in cycles; overexpression and/or deletion of single or multiple genes as may be desired.

In some embodiments of the disclosure, the recombinant cell for the high throughput methods is a bacterium, for example an E. coli. In other embodiments, the recombinant cell can be a yeast or fungal cell, e.g., a species of Saccharomyces (for example S. cerevisiae), Candida, Pichia, Schizosaccharomyces, Scheffersomyces, Blakeslea, Rhodotorula, Aspergillus or Yarrowia.

### Biosynthetic Methods

In some aspects, the disclosure provides a method for producing target proteins comprising: (i) generating a plurality of polyclonal genes in a plurality of expression vectors; (ii) generating a plurality of recombinant cells comprising the expression vectors; (iii) growing the plurality of recombinant cells under conditions effective to produce the target proteins; and (iv) isolating the target protein(s) from the recombinant cell. In some embodiments, the method comprises growing the recombinant cell under conditions effective to express the genes encoding the proteins, and for a period of time to produce adequate amounts of the target proteins.

Recombinant host cells expressing the target proteins can be grown, fermented, and produce proteins on various carbon and nitrogen sources as described herein or otherwise known in the art. Isolation and detection of products from recombinant cell or lysate can be performed by any methods described herein, or as otherwise known to those skilled in the art.

### Example Systems, Modular Workflow, and Methodology

The sequencing, interaction, and optimization of the methods, systems, and modules described herein allow rapid and scalable characterization of proteins with minimal need for human interventions. The systems and methods in accordance with the aspects and embodiments of the disclosure comprise custom, end-to-end software that drives the planning, executing, and analyzing of the experiments, while tracking and consolidating data recorded throughout the experimental protocol. The standardization that the software creates, for the first time, the opportunity to create additional machine learning algorithms that can improve the laboratory phase of de novo large and mid-scale protein production work, based on the high resolution and large number of datapoints provided by the methods and systems disclosed herein. These tools can help to identify features of designed proteins that can improve the overall design process. Further, these protocols can generate much more data in a standardized and searchable format compared to current standard methods produce less waste and require fewer reagents yet.

The aspects and embodiments relating to systems, methods, and/or modules described herein can comprise any variety of devices, computer programs, or instruments that are useful in operating the protein platforms and/or performing the methods for producing the target protein designs. In some embodiments the devices, programs, and/or instruments are automated or can be programmed for automatic control through a central processor on one or more computers. Some non-limiting examples of devices and instruments that fall within these aspects and embodiments include liquid handling devices, such as samplers, dispensers, autosamplers, autodispensers, pipettes and pipettors, acoustic liquid handlers, injectors, injector valves, injector loops, pumps, peristaltic pumps, and the like; filters and filter systems; mixers; switches, valves, and manifolds (e.g., vacuum manifold); robots (e.g., open source Opentrons, OT-2); chromatography systems such as HPLC, FPLC, preparative, semi-preparative, or analytic Size Exclusion Chromatography (SEC), ion-exchange chromatography, affinity chromatography, hydrophobic interaction chromatography, etc.; collection trays, fraction collectors, etc.; platforms and shakers for multi-well reaction or storage plates (e.g., 96 or 384 well plates); temperature control devices, such as heaters, dry block heaters, coolers, cooling blocks, incubators, thermostats, thermal cyclers, water baths, and the like; detectors (e.g., UV, UV-Vis, IR, radiation, fluorescence, phosphorescence, etc.); centrifuges; nucleic acid sequencing systems and analyzers (e.g., NGS, etc.); and mass spectrometers (e.g., LC/MS, LC/MS/MS, acoustic ejection-MS, TOF-MS, etc.); and custom codes and automatic scripts (e.g., python scripts) within electronic notebooks, databases, and/or dataframes.

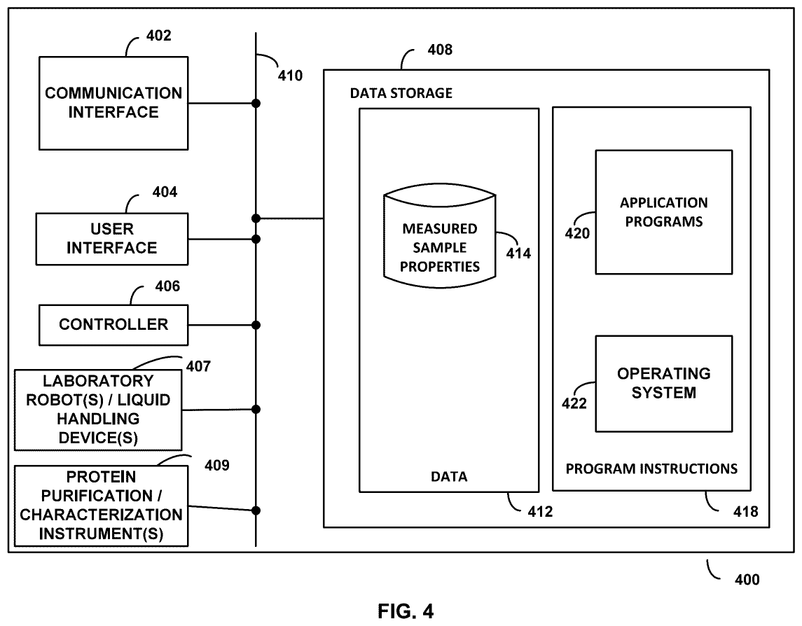

FIG. 4 illustrates an example automated laboratory system 400 that may be used to implement the methods described herein. By way of example and without limitation, system 400 may be include a computer (such as a desktop, notebook, tablet, or handheld computer, a server), elements of a cloud computing system, one or more robots (e.g., robotic pipettors or other laboratory robotic systems), a drone, an autonomous vehicle, or some other type of device. It should be understood that system 400 may represent a physical computing device such as a server, a particular physical hardware platform on which a laboratory applications operate in software, or other combinations of hardware and software that are configured to carry out functions as described herein.

As shown in FIG. 4, system 400 may include a communication interface 402, a user interface 404, a controller 406 (which may include one or more processors), one or more laboratory robot(s) and/or one or more liquid handling device(s) 407, one or more protein purification and/or characterization instrument(s) 409, and data storage 408, all of which may be communicatively linked together by a system bus, network, or other connection mechanism 410.

Communication interface 402 may function to allow system 400 to communicate, using analog or digital modulation of electric, magnetic, electromagnetic, optical, or other signals, with other devices, access networks, and/or transport networks. Thus, communication interface 402 may facilitate circuit-switched and/or packet-switched communication, such as plain old telephone service (POTS) communication and/or Internet protocol (IP) or other packetized communication. For instance, communication interface 402 may include a chipset and antenna arranged for wireless communication with a radio access network or an access point. Also, communication interface 402 may take the form of or include a wireline interface, such as an Ethernet, Universal Serial Bus (USB), or High-Definition Multimedia Interface (HDMI) port. Communication interface 402 may also take the form of or include a wireless interface, such as a Wifi, BLUETOOTH®, global positioning system (GPS), or wide-area wireless interface (e.g., WiMAX or 3GPP Long-Term Evolution (LTE)). However, other forms of physical layer interfaces and other types of standard or proprietary communication protocols may be used over communication interface 402. Furthermore, communication interface 402 may comprise multiple physical communication interfaces (e.g., a Wifi interface, a BLUETOOTH® interface, and a wide-area wireless interface).

User interface 404 may function to allow system 400 to interact with a user, for example to receive input from and/or to provide output to the user. Thus, user interface 404 may include input components such as a keypad, keyboard, touch-sensitive or presence-sensitive panel, computer mouse, trackball, joystick, microphone, and so on. User interface 404 may also include one or more output components such as a display screen which, for example, may be combined with a presence-sensitive panel. The display screen may be based on CRT, LCD, and/or LED technologies, or other technologies now known or later developed. User interface 404 may also be configured to generate audible output(s), via a speaker, speaker jack, audio output port, audio output device, earphones, and/or other similar devices.

Controller 406 may comprise one or more general purpose processors-e.g., microprocessors-and/or one or more special purpose processors-e.g., digital signal processors (DSPs), graphics processing units (GPUs), floating point units (FPUs), network processors, tensor processing units (TPUs), or application-specific integrated circuits (ASICs). Data storage 408 may include one or more volatile and/or non-volatile storage components, such as magnetic, optical, flash, or organic storage, and may be integrated in whole or in part with controller 406. Such storage could be used to store parameters that define the contents of the wells of one or more multi-well sample plates contained within the system 400, e.g., the identity of DNA sequences, vectors, cloned genes, target proteins and/or samples of polyclonal recombinant cells within a particular well have been modified to generate a target protein, an expected molecular weight or other property of such a target protein (e.g., concentration), a list of such target proteins and a mapping thereof to wells of multi-well sample plates contained in the system according to which of the wells contain samples modified to create each of the target proteins, or other information. Data storage 408 may include removable and/or non-removable components.

Controller 406 may be capable of executing program instructions 418 (e.g., compiled or non-compiled program logic and/or machine code) stored in data storage 408 to carry out the various functions described herein. Therefore, data storage 408 may include a non-transitory computer-readable medium, having stored thereon program instructions that, upon execution by system 400, cause system 400 to carry out any of the methods, processes, or functions disclosed in this specification and/or the accompanying drawings. The execution of program instructions 418 by controller 406 may result in controller 406 using data 412.

By way of example, program instructions 418 may include an operating system 422 (e.g., an operating system kernel, device driver(s), and/or other modules) and one or more application programs 420 installed on system 400. Such application programs 420 can include, e.g., functions for operating the robot(s)/device(s) 407 and instrument(s) 409 to prepare reaction (e.g., set temperatures, set times, withdrawal/dispense liquids, measure physical properties (e.g., turbidity, spectra, concentrations, molecular weights and/or yields) 414 of cells and/or proteins generated in respective wells of a multi-well sample plate, functions for operating the robot(s)/device(s) 407, based on such information 414, to consolidate into a single multi-well sample plate at least one sample of each protein of a set of target proteins (optionally at a normalized concentration), or other functions.

Application programs 420 may communicate with operating system 422 through one or more application programming interfaces (APIs). These APIs may facilitate, for instance, application programs 420 receiving sample measurements from the instrument 409, operating the robot(s)/device(s) 407 to transport samples between multi-well sample plates and/or to an input port of the instrument 409, transmitting or receiving information via communication interface 402, receiving and/or displaying information on user interface 404, and so on.

Application programs 420 may take the form of “apps” that could be downloadable to system 400 through one or more online application stores or application markets (via, e.g., the communication interface 402). However, application programs can also be installed on system 400 in other ways, such as via a web browser or through a physical interface (e.g., a USB port) of the system 400.

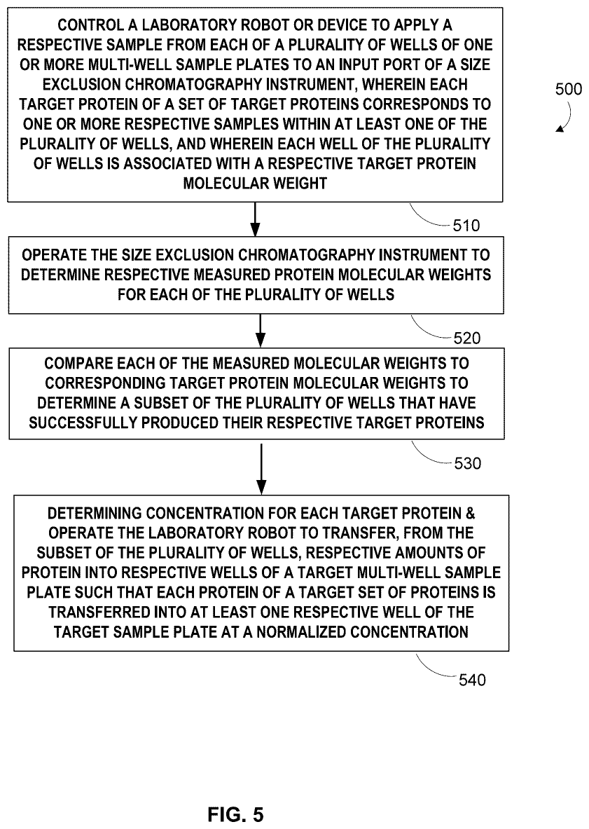

FIG. 5 is a flowchart of a set of operations 500 (e.g., of an “app” or other application program 420) that merely illustrate one set of exemplary operations that may be performed by a system (e.g., 400) as described herein. As shown, exemplary operations 500 include controlling a laboratory robot or device to apply a respective sample from each of a plurality of wells of one or more multi-well sample plates to an input port of a size exclusion chromatography instrument, wherein each target protein of a set of target proteins corresponds to one or more respective samples within at least two of the plurality of wells, and wherein each well of the plurality of wells is associated with a respective target protein molecular weight (510). The operations 500 additionally include operating the size exclusion chromatography instrument to determine respective measured protein molecular weights for each of the plurality of wells (520). The operations 500 yet further include comparing each of the measured molecular weights to corresponding target protein molecular weights to determine a subset of the plurality of wells that have successfully produced their respective target proteins (530). The operations 500 also include determining a concentration for each of the target proteins and operating the laboratory robot to transfer, from the subset of the plurality of wells, respective amounts of protein into respective wells of a target multi-well sample plate such that each protein of a target set of proteins is transferred into at least one respective well of the target sample plate at a normalized concentration (540). The operations 500 could include additional or alternative steps or features, in accordance with the various aspects and embodiments of the disclosure.

With respect to any or all of the message flow diagrams, scenarios, and flowcharts in the figures and as discussed herein, each step, block and/or communication may represent a processing of information and/or a transmission of information in accordance with example embodiments. Alternative embodiments are included within the scope of these example embodiments. In these alternative embodiments, for example, functions described as steps, blocks, transmissions, communications, requests, responses, and/or messages may be executed out of order from that shown or discussed, including in substantially concurrent or in reverse order, depending on the functionality involved. Further, more or fewer steps, blocks and/or functions may be used with any of the message flow diagrams, scenarios, and flow charts discussed herein, and these message flow diagrams, scenarios, and flow charts may be combined with one another, in part or in whole.

A step or block that represents a processing of information may correspond to circuitry that can be configured to perform the specific logical functions of a herein-described method or technique. Alternatively or additionally, a step or block that represents a processing of information may correspond to a module, a segment, or a portion of program code (including related data). The program code may include one or more instructions executable by a processor for implementing specific logical functions or actions in the method or technique. The program code and/or related data may be stored on any type of computer-readable medium, such as a storage device, including a disk drive, a hard drive, or other storage media.

The computer-readable medium may also include non-transitory computer-readable media such as computer-readable media that stores data for short periods of time like register memory, processor cache, and/or random access memory (RAM). The computer-readable media may also include non-transitory computer-readable media that stores program code and/or data for longer periods of time, such as secondary or persistent long term storage, like read only memory (ROM), optical or magnetic disks, and/or compact-disc read only memory (CD-ROM), for example. The computer-readable media may also be any other volatile or non-volatile storage systems. A computer-readable medium may be considered a computer-readable storage medium, for example, or a tangible storage device.

Moreover, a step or block that represents one or more information transmissions may correspond to information transmissions between software and/or hardware modules in the same physical device. However, other information transmissions may be between software modules and/or hardware modules in different physical devices.

The following Examples illustrate several aspects and embodiments in accordance with the disclosure and in no way serve to limit the scope or spirit of the claims.

### EXAMPLES

### Example 1—High Throughput Protein Design, Production, and Characterization

### Gene Design and Synthesis

Target protein sequences or structures in Protein Data Bank (pdb) format are converted to DNA sequences using an automated executable script, rather than manual conversion from codon usage tables. The script is capable of generating sequences that (i) are codon-optimized for expression in multiple organisms (E. Coli, human, yeast, etc.), (ii) prevent alternative start sites in the genes, and (iii) are optimized for DNA synthesis. The necessary DNA overhangs for cloning are added onto the genes, which are then formatted into appropriate format for synthesis either on a DNA synthesizer or as an output file for a third party DNA manufacturer/supplier. The script can automatically execute a number of quality checks (e.g., no duplicate sequences, sequence length for multi-fragment assembly, synthesizability of sequences using manufacturer API).

### Plasmid Construction

Plasmids for expressing proteins are constructed from synthetic DNA according to the following procedure: Linear DNA fragments (Integrated DNA Technologies, IDT eblocks) encoding design sequences, and including overhangs that are suitable for a Bsal restriction digest are cloned into custom target vectors using Golden Gate Assembly. Entry vectors are created to support a variety of N-and C-terminal tags. For example a standard expression vector used for screening designs is as C-terminally HIS-tagged constructs: MSG-design-GSGSHHWGSTHHHHHH (SEQ ID NO: 4, entry vector LM627), where the underlined sequence is the SNAC-tag (20) used for cleaving the HIS-tag (cleaving not used in this work), and also contains a TRP residue to ensure proteins have measurable absorbance at 280 nm.

For this example, the entry vectors are engineered for Golden Gate cloning as modified pET29b+ vectors that contain a lethal ccdb gene positioned between the BsaI restriction sites that is both under control of a constitutive promoter and in the T7 promoter reading frame. The lethal ccdb gene reduces background by ensuring that plasmids that do not contain an insert (and therefore still carry the lethal gene) kill those transformant cells. The vectors are propagated in ccdb resistant NEB Stable cells (New England biolabs C3040H, grown from fresh transformant cells).

The Golden Gate cloning reactions (1 uL per well) are set up on a 96 well PCR plate using an ECHO acoustic liquid handler (Labcyte ECHO 525, Beckmann Coulter):

| Amount | | Reagent |
|---|---|---|
| 0.1 | uL | 10× T4 Ligase Buffer |
| 3 | Units | BsaI-HFv2 restriction enzyme |
| 100 | Units | T4 DNA Ligase |
| 3 | fmol | Target GGA vector |
| 6 | fmol | Linear DNA insert |

| | |
|---|---|
| Fill to 1 μL | Cloning-grade H2O |

Alternatively, when not using a liquid handler such as an ECHO device, the Golden Gate cloning reactions can be scaled up to 5 uL volume and pipetted by hand, using 12-channel pipettes. The reactions are incubated at 37° C. for 20 minutes, followed by 5 min at 60° C. (IKA Dry Block Heater 3).

### Protein Expression and Purification

For experimental screens, Golden Gate reaction mixtures are transformed directly into BL21(DE3) (New England Biolabs) as follows: 1 uL of reaction mixture is incubated with 6 uL of competent cells on ice in a 96 well PCR plate. The mixture is incubated on ice for 30 minutes, then heat-shocked for 10 seconds at 42° C. in a block heater (IKA Dry Block Heater 3), then rested on ice for 2 minutes. Subsequently, 100 uL of room temperature SOC media (New England Biolabs) is added to the cells, followed by incubation at 37° C. with shaking at 1000 rpm in an incubator (Heidolph Titramax1000/Incubator 1000).

The transformed cells are grown in a 96 well deep-well plate (2 mL total well volume) in autoclaved LB media supplemented with 50 μg/mL Kanamycin at 37° C. and 1000rpm. All growth plates are covered with breathable film (Breathe Easier, Diversified Biotech) during incubation. The following day, glycerol stocks can be made from the overnight LB cultures (100 uL of 50% [v/v] Glycerol in water mixed with 100 uL bacterial culture, frozen and kept at −80° C.

Expression cultures are seeded from the LB plate or directly from the recovered transformed cells in SOC media as 4×1 mL cultures in 96 well deep-well plates of TB-II Autoinduction media (TBII-5052) with the appropriate antibiotic (50 μg/mL Kanamycin). TBII-5052 includes Terrific Broth II (MP biomedicals, a TRIS-buffered, rich media made according to manufacturer's specifications) supplemented with 2 mM Magnesium Sulfate (2 mL in 1 L from 1 M stock), and 1× dilution (20 mL in 1 L) of a 50×5052 autoinduction buffer (50× made as: 250 g glycerol, 25 g D-glucose, 100 g D-lactose monohydrate in 1 L of ultrapure water). Cultures are grown at 1000 rpm at 37° C. for at least 20h before harvest.

The cells are harvested by centrifugation at 4000×g for 5 min. Growth media is discarded by rapidly inverting the plate, and harvested cell pellets are either processed directly, or frozen at −80° C. Bacterial pellets are resuspended and lysed in 100 uL per 1 mL of culture volume B-PER chemical lysis buffer (Thermo Fisher Scientific) supplemented with 0.1 mg mL−1Lysozyme (from a 100 mg mL−1 stock in 50% [v/v] Glycerol, kept at −20° C., Millipore Sigma), 50 Units of Benzonase per mL (Merck/Millipore Sigma, stored at −20° C.), and 1 mM PMSF (Roche Diagnostics, from a 100 mM stock kept in Propan-2-ol, stored at room temperature). The four growth plates with the lysis buffer are sealed with an aluminum foil cover and shaken for 5 minutes at 1000 rpm, before being consolidated into a single 96 w plate, and spun down at 4000×g for 15 minutes.

For purification of proteins using HIS tag-based Immobilized metal affinity chromatography (IMAC), 50 uL of Nickel-NTA resin bed volume (Thermo Scientific) is added to each well of a 96 well fritted plate (25 μm frit, Agilent 200953-100). The resin is regenerated before each run and stored in 20% [v/v] Ethanol). To increase wash step speed, the resin is equilibrated on a plate vacuum manifold (Supelco, Sigma or Pall Biosciences or Macherey Nagel) by drawing 3×500 uL of Wash buffer (20 mM Tris, 300 mM NaCl, 25 mM Imidazole, pH 8.0) over the resin using the vacuum manifold at its lowest pressure setting. The supernatant of the lysate (approximately 400 uL per well) is extracted after the spin down and applied to the equilibrated resin and allowed to slowly drip through over ˜5 minutes. Subsequently the resin is washed on the vacuum manifold with 3×500 uL per well of Wash buffer. The fritted plate spouts are blotted on paper towels (Kimwipes, Kimberly-Clark) to drain excess Wash buffer. To each well is added 200 uL of Elution buffer (20 mM Tris, 300 mM NaCl, 500 mM Imidazole, pH 8.0) and incubated for 5 minutes before eluting the protein by centrifugation at 1000×g for 5 minutes into a 96 sterile filter plate (0.2 μm, Agilent 203940-100), followed by a 96 well collection plate. Eluate is stored at 4° C., or directly processed by size exclusion chromatography (SEC).

### Size Exclusion Chromatography

The de novo protein designs are subject to a solubility screen by size exclusion chromatography (SEC), using S75 or S200 5/150 columns (Cytiva) at up to 0.65 mL/min flow rate in 20 mM Phosphate, 100 mM NaCl at pH 7.4 or Phosphate Buffered Saline on an Akta pure (Cytiva) with an autosampler module, or a Agilent 1260 Infinity II bioinert system. Absorbance was monitored at 280 nm. All protein designs and buffers are sterile filtered through 0.2 micrometer filters before being run through the columns. Fractions are collected in 96 or 384 deep well plates.

### Data Processing and Robotic Sample Pooling

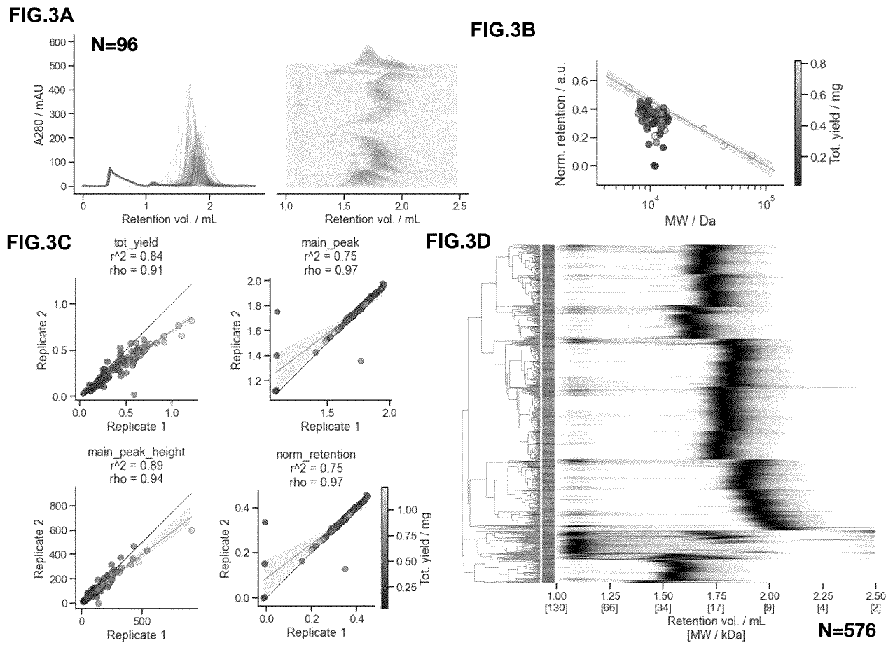

The size exclusion chromatography (SEC) data is processed through an interactive Jupyter notebook, which consolidates all recorded data and calculates the expected properties of expressed protein designs, (e.g., molecular weights). From SEC trace peaks at defined retention volumes the oligomeric state of the proteins can be inferred, as well as yield determined by integrating the absorbance at 280 nm. The analysis software compares the measured molecular weights (from SEC, assigned by running known calibration standards, Cytiva LMW and HMW calibration kits) with the expected values and selects appropriate fractions for pooling. The software then sends instructions for the OT-2 robot, which pools the selected fractions into a single 96 or 384 well plate. The OT-2 robot can also normalize concentrations using the integrated A280 absorbance data from the SEC traces. The workflow provides a final plate containing all designs at known concentrations with all associated data and is prepared within 48 hours of receiving the linear DNA for generating the synthetic genes. All information on the proteins tested, as well as experimental data is consolidated in a single dataframe that can be used for downstream analysis. This protocol also reduces the bench time for the researcher to about 3 h. The results of this run are depicted in FIG. 3.

### Example 2

### Gene Design and Synthesis

Protein sequences are first reverse-translated to DNA sequences using a custom python script that takes a FASTA file or a list of PDBs as input. Codon optimization is performed with DNA chisel (python library optimizer) using a mixed objective function that optimizes user-defined codon adaptation index (CAI), minimizes repeat sequences, avoids BsaI restriction endonuclease recognition sites, and suppresses alternative start sites. The script also ensures synthesizability on the fly by using IDT's SciTools Plus API, and if the original sequence cannot be made, the objective function is gradually modified by up-weighting the repeat sequence cost parameter until synthesizability is achieved. If the reverse-translated sequence is longer than a user-specified length (default: 1500 bp, IDT's eBlock maximum), the sequence is automatically split into two fragments for one-pot Golden Gate Assembly (GGA). The 4 bp fragment overhang is chosen from a predefined list that maximizes orthogonality to the entry vector GGA sites (AGGA and TTCC) in order to ensure assembly fidelity. Finally, overhangs for GGA containing Bsal recognition sites are appended to the 3′ and 5′ ends, and random filler sequences added to the outer parts if the sequence is less than 300 bp (IDT's eBlock length minimum). The script outputs a formatted spreadsheet that is directly uploaded to IDT for ordering as eBlocks, and an ECHO transfer protocol for automated cloning (see below).

DNA fragments are ordered resuspended in IDTE buffer (10 mM Tris, 0.1 mM EDTA, pH 8.0) in either 96-well PCR plates (for manual cloning), or ECHO-qualified 384-well plates (for robotic-enabled cloning). The whole process is fully automated, and going from a list of amino acid sequences or PDBs to placing the gene fragment order only takes minutes.

The output provided by the script is a dataframe of all DNA sequences to order, expected sequences on the target plasmid, and an ordering spreadsheet or csv file that can be uploaded to the manufacturer (e.g., IDT, Twist, or others). Optionally, the DNA fragments can be concentration normalized depending on cloning and gene construction methods (e.g., provided in Labcyte ECHO compatible 384 well plates).

### Plasmid Construction

Similar to the Example above, vectors are propagated and produced in bulk from ccdB-resistant NEB Stable E. coli chemically competent cells (New England Biolabs). Following transformation according to the manufacturer's protocol (see below), the transformants are grown overnight at 37° C. on LB-Agar containing 50 μg/mL of kanamycin sulfate. Single colonies are used to inoculate 50 mL of liquid LB cultures (in 250 mL baffled conical flasks) containing 50 μg/mL of kanamycin sulfate, and grown overnight at 37° C. with shaking at 250 rpm. Plasmids are purified using a ZymoPURE II Plasmid Midiprep Kit (Zymo Research) according to the manufacturer's protocol, and verified by full plasmid sequencing (Plasmidsaurus). Typical yields are 50-100 μg, which is enough for ˜2,000-4,000 GGA reactions.

GGA reactions are assembled with an ECHO 525 acoustic liquid handling robot (Beckman Coulter) by using the transfer protocol outputted during DNA fragment generation. A master mix containing Bsal-HFv2 (New England Biolabs), T4 DNA ligase (New England Biolabs), T4 DNA ligase buffer (New England Biolabs) and the entry vector is prepared first, and the ECHO is used to combine master mix and DNA fragments into 96-well PCR plates (1 μL final volumes). Reactions are incubated at 37° C. for 15 min, followed by 5 min at 60° C. GGA products are directly transformed into an E. coli expression strain by adding 6 μL of BL21 (DE3) chemically competent cells (New England Biolabs) to the 1 μL reactions. Transformations are performed in 96-well PCR plates according to the manufacturer's protocol (see below), and the transformed cells are used to inoculate expression cultures directly (transformation efficiency=858±443 CFUs (mean±standard deviation, N=12)). Going from linear DNA to inoculated cultures takes about 2 h for 192 GGA reactions, and only an extra ˜15 min for every additional 96-well plate.

More specifically, for the GGA reaction a ratio of target vector to linear DNA fragment of 1:2 respectively, provides a twofold molar excess of insert to target vector. When using an ECHO device, a 1 μL total reaction volume can be used, based on an interactive spreadsheet calculator that takes into account all parameters to achieve an optimal reaction mixture. For this example, the recipe (for NEB enzymes) uses the following ratios in a total volume of 1 μL:

| Amount | | Reagent |
|---|---|---|
| 0.1 | uL | 10× T4 Ligase Buffer |
| 1.2 | Units | BsaI-HFv2 restriction enzyme |
| 40 | Units | T4 DNA Ligase |
| 2-4 | fmol | Target GGA vector |
| 4-8 | fmol | Linear DNA insert |

| | |
|---|---|
| | (at twofold excess to GGA target vector) |
| Fill to 1 μL | Cloning-grade H2O |

When assembling the GGA reactions with multichannel pipettes or individual tubes by hand at 5 μL total reaction volume another interactive spreadsheet calculator is used. The recipe (for NEB enzymes) for this protocol in a total volume of 5 uL:

| Amount | | Reagent |
|---|---|---|
| 0.5 | uL | 10× T4 Ligase Buffer |
| 3 | Units | BsaI-HFv2 restriction enzyme |
| 100 | Units | T4 DNA Ligase |
| 20-30 | fmol | Target GGA vector |
| 40-60 | fmol | Linear DNA insert |
| | | (at twofold excess to GGA target vector) |

| | |
|---|---|
| Fill to 5 μL | Cloning-grade H2O |

For each of these protocols, the reactions are assembled into 96 W PCR Plates, and spun down briefly (1000×g, 30 s) to collect reaction mixture at the bottom of each well, then covered with aluminum or transparent foil, and incubated at 37° C. for 20 minutes. If desired, reaction mixtures can be inactivated by incubation at 60° C. for 5 minutes, and subsequently spun down (1000×g, 30 s).

Alternatively, custom GGA target vectors for expression in E. Coli DE3 strains can be made using a template vector such as LM1369 described herein. This general template vector is a pET backbone that features PaqCI Type IIs restriction cloning sites for GGA assembly at the start and stop codons, allowing insertion of linear DNA fragments of arbitrary N-and/or C-terminal tags with a ccdb insert to facilitate creation of customized target GGA vectors for the cloning strategy described above. A custom GGA target vector can thus be comfortably assembled in 3 days (Day 1 GGA with LM1369 and plating, Day 2 picking clones for overnight growth, Day 3 DNA prep and sequencing)—excluding time for overnight sequence confirmation. We provide a spreadsheet calculator for the assembly of PaqCI GGA.

For transformation and protein expression, all steps are performed under flame. Further competent cells are maintained as cold as possible, and are dispensed directly after they have been thawed-avoiding delays aids in the success of the methods.

For transformation into the E. Coli expression strain BL21 (DE3) 1 μL of the GGA reaction mixture is used. When using the ECHO device, the 96 W PCR Plate with 1 μL of GGA reaction is directly placed on ice. When using the alternative 5 μL pipette reaction approach 1μL of GGA reaction is transferred to the bottom of each well of a fresh 96W PCR plate, which is placed on ice. Given that these transformations are in small volumes and produces on the order of hundreds of CFUs, when using less competent cells below the transformation efficiency of commercial cell lines (around 107 CFUs/μL of pUC19 vector), cell suspension volume should be increased accordingly.

Transformation. Competent BL21 (DE3) cells (6 μL) are transferred into each well of the 96 w PCR plate using a precooled (stored at −20° C.) dispenser tip of 200 μL total volume on an electronic repeater pipette, typically three 200 μL aliquots of NEB BL21 (DE3) for one 96 w plate. The pipetting of cells requires careful handling as the cell suspension releases poorly from the pipette tip. Dispensing onto the side of each well, aligning the pipette almost horizontally, and retracting the tip orthogonal to the well aids in releasing the volume of cells.

To aid with mixing of the GGA reaction product and the competent cell drops, the 96 w PCR plate is rapidly moved downward to “hand centrifuge” the mixture to the bottom of each well. The competent cells with the GGA mixture are incubated on ice for 30 min while covered (e.g., plastic lid or aluminum foil). The cells are then heat shocked by placing the 96 w PCR plate in a dry block heater at 42° C. for 10 seconds, and then back on ice for 2 minutes. Subsequently using an electronic repeater, 100 μL of room temperature SOC media is added to each well, covered with breathable film, and the plate incubated at 37° C. shaking at 1000 rpm on an orbital plate shaker for 1 hour.

Protein expression/autoinduction. In overview, protein expression is performed in round bottom 96 deepwell plates filled with 1 mL of simplified auto-induction media (TB II, MP Biomedicals, supplemented with glycerol (5 g/L), glucose (0.5 g/L), lactose (2 g/L), MgSO₄ (2 mM), and kanamycin sulfate (50 μg/mL)). Transformation reactions are split 4-fold and used to inoculate 4×1 mL of expression cultures directly, followed by incubation at 37° C. for 20 h at 1,000 rpm on a 2.5 mm orbital shaker. The induction media can be prepared in advance and stored at 4-8° C. for about 2-3 weeks.

Under flame, 1 mL of media is dispensed per well into four 96 w round bottom deep well plates for protein expression using a 1 mL multichannel pipette. The number of expression culture plates can be increased if more protein produced is desired.

Transformed cells, after recovery, are transfer into each well (25 μuL per well) of the 96 w round bottom deep well plates for expression and mixed with 1 mL of autoinduction media. The plates are covered with breathable film and incubated for at least about 20 hours at 37° C. on an orbital plate shaker (longer times may be needed in order to ensure the cultures induce). The cells are harvest as pellets by centrifuging at 4000×g for 5 minutes, and the supernatant is removed and discarded. The plates are covered with aluminum foil and can stored at −80° C. or can proceed directly to purification.

Glycerol stocks of the cultures can be prepared by transferring transformed cells, after recovery, into a 96 w round bottom deep well plate containing 1 mL LB media per well with appropriate antibiotic, covered with breathable film, and incubated for 1 h at 37° C., shaking at 1000 rpm on an orbital plate shaker. After, 50 uL of the cells are dispensed into each expression plate to be incubated for 20 h as above. The cells are allowed to grow in the LB media plate overnight (12-16 h) at 37° C. at 1000 rpm on an orbital plate shaker. Glycerol stocks are prepared in a 96 w receiver plate by adding, per well, 100 μL of sterile 50% (v/v) Glycerol in H2O+100 μL cultures in LB media (swirl pipette tips in wells to mix) and covered with aluminum foil to be stored at −80° C.

Protein purification. In overview, cells are harvested in 96 deepwell plates by centrifugation (4,000×g, 5 min), the media discarded, and cells resuspended in lysis buffer (B-PER, Thermo Fisher Scientific, supplemented with lysozyme [0.1 mg/mL], PMSF [1 mM], Benzonase [25 U/mL], 100 μL per 1 mL of culture-equivalent pellet). Lysis is left to proceed for 15 min at 37° C. under agitation (1,000 rpm) before pooling the lysates (4×100 uL) into one 96 deepwell plate and clearing debris by centrifugation (4,000×g, 15 min). Proteins are purified from the soluble fraction by binding to Ni-NTA resin (50 μL of resin bed per well, added to 96-well fritted plates (25 μm PE frit, Agilent Technologies)), followed by three wash cycles using a plate vacuum manifold (3×400 μL, 20 mM Tris, 300 mM NaCl, 25 mM imidazole, pH 8.0). Proteins are eluted from the resin by addition of 200 μL of elution buffer (20 mM Tris, 300 mM NaCl, 500 mM imidazole, pH 8.0), and sterile-filtered by centrifugation into a 96-well filter plate (0.22 μm pore-size) mounted on a receiver plate. The process is designed to take under 2 h from cell harvest to injection-ready eluates for up to two 96-well plates, with only minimal extra time necessary for additional plates. The final output provides the 192 samples in elution buffer, sterile filtered in two 96 w receiver plates, and which are ready for SEC.

Once purified, or while bound to the affinity resin, any affinity tags (i.e., the His-tag) can be removed. To do so, the protocol can be modified to add two additional wash steps (500μL per well) using SNAC cleavage buffer, following the initial wash steps above. In this pate format, the spouts of the fritted plate are blotted, and the bottom of the fritted plate is sealed using parafilm: A fresh sheet of parafilm is placed over a new 96 w deep well plate, and the fritted plates spout is pushed first onto the flat parafilm covering the new plate to align the spouts and achieve a watertight seal. SNAC buffer with 2 mM NiCl₂ is added to initiate the cleaving on resin. To top of the wells of the fritted plate are dried and sealed using aluminum foil, and incubated with shaking overnight. After at least 16 h of incubation at RT or 37° C. for improved efficiency, the flow through is collected, as in the protocol above and is ready for SEC.

### Size Exclusion Chromatography

For the size exclusion chromatography in this example, separations are performed on 3 mL resin bed volumes columns, Cytiva Superdex 5-150 GL either S75 (3-70 kDa) or S200 (10-600kDa) depending on expected candidate protein size. To process the sample sizes in this example, (approximately 288 overnight, typically limited by fraction collector capacity) an autosampler that can automatically draw and inject hundreds of samples onto the chromatography systems is used. Two methods using two different instruments have been developed to execute the runs, a Cytiva Akta pure with autosampler, and an Agilent 1260 bioinert with multisampler.

Agilent 1260. The multisampler module on the Agilent 1260 and 1290 FPLC series allows the injection of the samples automatically overnight. The loop volume for injection should be at least 100 μL. Samples are injected sequentially at flow rates of 0.65 mL/min. Maintaining pump pressures of 40-60 bars on S200 and S75 5-150 Superdex models.

Akta pure. The Akta pure system is equipped with an autosampler module to inject the samples automatically. To achieve low dead volume when injecting from the autosampler, the Akta flow path was re-wired. The samples are directly injected from the autosampler onto the SEC column, which then bypasses the column valve and connects directly to the UV detector. To minimize the dead volume between the UV detector and the fractionation arm, the tubing of the fractionator is switched to “blue” i.d. 0.25 mm.

### Data Processing

The SEC chromatograms are batch-analyzed by a custom python script using NumPy (Harris, C.R., et al. Array programming with NumPy. Nature 585, 357-362 (2020)), SciPy (Pauli Virtanen, et al., and SciPy 1.0 Contributors. (2020) SciPy 1.0: Fundamental Algorithms for Scientific Computing in Python. Nature Methods, 17(3), 261-272.), and Pandas (Mckinney, W., et al., (2010). Data structures for statistical computing in python. In Proceedings of the 9th Python in Science Conference (Vol. 445, pp. 51-56)), to detect peaks in the protein absorbance signal at 280 nm and convert their retention volume to approximate molecular weight compared to calibration curve assembled from standards (LMW [S75], or HMW [S200], Cytvia). The script also generates code to program an OT-2 liquid handling robot (Opentrons) for automated picking, pooling and normalizing fractions. The opentrons script includes a custom function of extract from 384 well plates with 250 uL internal volumes with 300 microliter pipette tips in two pipetting steps to avoid displacing and overflowing the liquid from a well through the added volume of the pipette tip, the extraction from a well is achieved in two consecutive aspiration steps where only the second step aspirates from the bottom of the well. By integrating the absorbance signal to determine protein concentration in each fraction, information can be extracted for each design including, total soluble yield, polydispersity, estimated molecular weight and aggregation state of each peak, and desired fractions to pool. The data is exported into a standardized open format dataframe (HDF5 from The HDF Group. Hierarchical Data Format, version 5, 1997-2024.). The end-point of the protocol provides a set of 96-well plates containing SEC-purified and concentration-normalized proteins (at a few hundred microliters at single to double-digit micromolar concentrations).

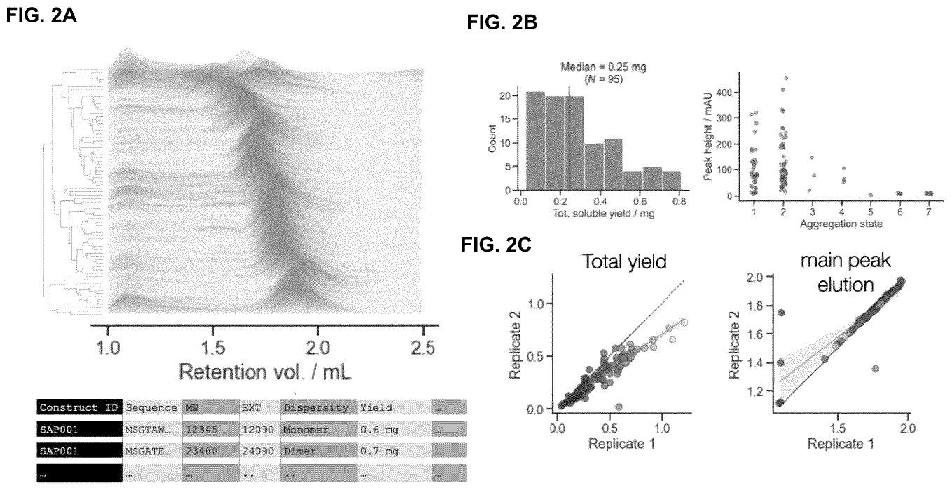

The results of this run are depicted in FIG. 2.

### Mass Spectrometry

Intact mass spectra were obtained by reverse-phase LC/MS on an Agilent G6230B TOF using an AdvanceBio RP-Desalting column (A: H2O with 0.1% Formic Acid, B: Acetonitrile with 0.1% Formic Acid), and subsequently deconvoluted with Bioconfirm using a total entropy algorithm. Protein masses were determined with a resolution of 1 Dalton.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall control. The foregoing detailed description and examples have been given merely for illustration and clarity of understanding. No unnecessary limitations are to be inferred or understood therefrom. The disclosure and claims are not limited to the exact details shown and described, as variations obvious to one skilled in the art will be included within the scope of the disclosure as well as the claims.

1. A semi-automated protein production platform comprising a polyclonal expression system that comprises a plurality of cloning products, wherein each of the plurality of the cloning products comprises a gene encoding a protein target and a background suppression vector, wherein the plurality of cloning products are adapted for transformation to generate recombinant cells, and wherein the generated recombinant cells are capable of direct expression of the protein targets.

2. The semi-automated protein production platform according to claim 1, further comprising a laboratory robot and/or a liquid handling device.

3. The semi-automated protein production platform according to claim 2, wherein the laboratory robot or liquid handling device comprises an OT-2 robot, an autopipettor, a vacuum manifold, a liquid chromatography machine, a robotic pipettor, or an acoustic liquid handler.

4. The semi-automated protein production platform according to claim 1, further comprising at least one chromatography column selected from an affinity column, an ion exchange column, a hydrophobic interaction column, a size exclusion column, or any combination of two of more thereof.

5. A semi-automated protein production method comprising, in a continuous workflow sequence,
identifying at least one target protein sequence to be produced by the method;
preparing at least one cloning reaction wherein the at least one cloning reaction comprises constructing a target gene sequence from one or more DNA sequences that encodes the at least one target protein sequence, in combination with a vector that comprises background suppression, to generate at least one cloning reaction product,
transforming cells with the at least one cloning reaction product from the at least one cloning reaction to generate a recombinant cell, and inducing the recombinant cell to express the target protein.

6. The method according to claim 5, further comprising purifying the target protein from the recombinant cell, wherein the purifying comprising at least one of an affinity column or resin, an ion exchange column or resin, a hydrophobic interaction column or resin, a size exclusion column or resin, or any combination of two of more thereof.

7. The method according to claim 5, further comprising determining the molecular weight of the target protein, wherein determining the molecular weight comprises at least one of size exclusion chromatography, mass spectrometry, or gel electrophoresis.

8. The method according to claim 6, further comprising determining the concentration of the purified target protein from a signal from an instrument associated with the column or resin.

9. The method according to claim 8, further comprising dispensing an amount of the purified target protein into a vessel, wherein the amount of purified protein that is dispensed is in a volume that provides for a known normalized concentration.

10. A non-transitory computer-readable medium having computer-executable instructions stored thereon that, if executed by one or more processors of a computing device, cause the computing device to perform operations comprising:
controlling a laboratory robot to apply a respective sample from each of a plurality of wells of one or more multi-well sample plates to an input port of a device or instrument, wherein each target protein of a set of target proteins corresponds to one or more respective samples within at least one of the plurality of wells, and wherein each well of the plurality of wells is associated with at least one expected measurable characteristic or value associated with the respective target protein;
operating the device or instrument to determine a characteristic or value associated with the protein for the at least one of the plurality of wells;
comparing each of the measured characteristic or value to the corresponding expected measurable characteristic or value to determine a subset of the plurality of wells that contain successfully produced target protein; and
operating the laboratory robot to transfer, from the subset of the plurality of wells, respective amounts of protein into respective wells of a target multi-well sample plate such that each protein of a target set of proteins is transferred into at least one respective well of the target sample plate.

11. The non-transitory computer-readable medium of claim 10, wherein operating the device or instrument comprises a size exclusion chromatography instrument to determine respective measured protein molecular weights for each of the plurality of wells.

12. The non-transitory computer-readable medium of claim 11, wherein the size exclusion chromatography instrument is operated such that samples from more than one of the plurality of wells are resident within the size exclusion chromatography instrument at a time.

13. The non-transitory computer-readable medium of claim 11, wherein operating the size exclusion chromatography instrument further comprises determining respective measured protein yields and/or concentrations for each of the plurality of wells, and wherein operating the laboratory robot further comprises transferring from the one or more wells respective amounts of protein into a particular well of the target multi-well sample plate such that an amount and/or a concentration of protein in the particular well corresponds to a target concentration.

14. The non-transitory computer-readable medium of claim 12, wherein operating the size exclusion chromatography instrument to determine respective measured protein yields for each of the plurality of wells comprises, for each of the plurality of wells, determining an integrated absorbance at 280 nanometers.

15. The non-transitory computer-readable medium of claim 10, wherein operating the device or instrument comprises a mass spectrometer to determine respective measured protein molecular weights for each of the plurality of wells.

16. A non-transitory computer-readable medium having computer-executable instructions stored thereon that, if executed by one or more processors of a computing device, cause the computing device to perform operations comprising:
operating a laboratory robot and/or liquid handling device to prepare one or more cloning reactions by dispensing a respective sample from each of a plurality of wells to form one or more reaction volumes, wherein each of the one or more reaction volumes corresponds to at least one target protein sequence, and wherein the samples forming the reaction volume are dispensed from (i) at least one of the plurality of wells comprising one or more DNA sequences that encodes the at least one target protein sequence, and (ii) at least one of the plurality of wells comprising a background suppression vector, to generate at least one cloning reaction product in each of the one or more reaction volumes;
operating a laboratory robot and/or liquid handling device to transfer a volume of competent cells to each of the one or more reaction volumes comprising the cloning reaction product to form a transformation mixture;
maintaining the transformation mixture under conditions that allow for transformation of the competent cells to generate recombinant cells comprising the cloning reaction product; and
maintaining the recombinant cells under conditions that allow for the recombinant cell to express the target protein.

17. The non-transitory computer-readable medium of claim 16, further comprising operations effective to maximize the yield of the one or more cloning reactions for a correct target plasmid and/or to reduce background plasmids comprising a failed cloning reaction.

18. The non-transitory computer-readable medium of claim 17, wherein the operations effective to maximize the yield of a cloning reaction one or more cloning reactions comprise selecting (i) a background suppression vector comprising a ccdb lethal gene and/or (ii) the one or more DNA sequences comprise overhangs designed for simultaneous and directional assembly of the DNA sequences into a single DNA sequence using a Type IIS restriction enzyme and T4 DNA ligase, and optionally verifying products of the cloning reactions by at least one of DNA sequencing or mass spectrometry.
